(19) United States
(12) Patent Application Publication
(10) Pub. No.: US 2021/0315729 A1
(43) Pub. Date: Oct. 14, 2021

(54) DENTAL ORAL APPLIANCE

(71) Applicant: Chi-Ching Huang, Taoyuan City (TW)

(72) Inventors: Chi-Ching Huang, Taoyuan City (TW); Tzu-Yun Kao, Taoyuan City (TW)

(21) Appl. No.: 16/923,175

(22) Filed: Jul. 8, 2020

(30) Foreign Application Priority Data
Apr. 14, 2020 (CN) 202020542871.7

(51) Int. Cl. A61F 5/56

(52) U.S. Cl. CPC A61F 5/566

(57) ABSTRACT

A dental oral appliance for using inside an oral cavity, where the oral cavity has an upper row of teeth, a lower row of teeth, a tongue, and an upper jaw. The upper and lower row of teeth define a lingual side. The dental oral appliance includes a separating portion and a tongue supporting portion, where an upper teeth region is defined above the separating portion and a lower teeth region is defined underneath thereof. The upper teeth region is adjacently disposed around the upper row of teeth, and the lower teeth region is adjacently disposed around the lower row of teeth. The outer edge of the tongue supporting portion is connected to one side of the separating portion close to the tongue.

### CROSS-REFERENCE TO RELATED APPLICATION

This application claims priority to and the benefit of Chinese Patent Application No. 202020542871.7 filed in the National Intellectual Property Administration, China on Apr. 14, 2020, the entire contents of which are incorporated herein by reference.

### BACKGROUND OF THE INVENTION

### Field of the Invention

The present invention relates to a dental oral appliance, more particularly a dental oral appliance capable of teeth securing, muscle correction, nose breathing training, and snoring reduction to improve sleep quality.

### Description of Related Art

So many people have problem of snoring, sleeping interruption or mouth breathing while sleeping, but they sometimes pay minimal attention on these issues. Actually, temporary stoppage of breathing caused from snoring will normally end up with sleep apnea syndrome. When human respiratory passage is blocked, there won't be enough air reaching human's lung, and thus anoxia or hypoxia is occurred, as well as carbon dioxide level within human body begins to rise. Which will lead to human waking up abruptly, accompanying with choking or gasping for air. Afterward, human's respiratory tract reopens, and then the patient is likely to take several deep breaths and falls back to sleep. Soon after, another round of pauses in breathing may follow, and the cycle will repeat itself throughout whole night. This would cause the sleep broken into several pieces. If human have both snoring, mouth-breathing and obstructive sleep apnea, it would cause long term anoxia or hypoxia, therefore the blood vessels become narrower, and then the damage is easily made. Which can bring several health issues such as high blood pressure, fatigue, cardiovascular disease, dysautonomia, and dysglycemia. As time goes by, these problems can cause further and unexpected damages to the heart, kidney, and other organs, while increasing the chances of sudden death. In addition, some people have problem of mouth breathing, which probably results into respiratory allergy, lower resting position of the tongue, adenoids, or hyperplasia of tonsils. At some worst cases, these symptoms could further lead to malfunction of the tongue, difficulty of swallowing, gum disease, teeth dislocation, bone deformities, or nasal septum deviation, etc. All of these problem could negatively impact one's sleep quality and physical well-being.

There are several causes of snoring, the most common one is that: the muscle holds and supports the throat relaxes while sleep, causing oral muscles to retreat and making the tongue to drop below its normal resting position; in turn, human's respiratory tract becomes narrower and then air flows faster, making the soft palate, uvula, tongue, and tonsils in a relaxed state to vibrate with big noise. For mouth-breathing, the causes might also include cold, allergy, nasal polyp, and some structural problem such as tongue-tie (ankyloglossia) and other oral or nasal cavity.

In view of the foregoing the present objective, it is desirable to gradually improve sleep quality while reducing snoring and mouth-breathing symptoms, through a sustainable, non-surgical, and non-invasive treatment that allows the patient to self-adjust and self-correct his/her sleep.

### SUMMARY OF THE INVENTION

The first objective of the present invention is to have non-surgical and non-invasive treatment to address the issue of bad quality sleep.

The second objective of the present invention is to adjust and correct severe snoring, lower position tongue and mouth breathing, through a gradually and mitigated approach.

The third objective of the present invention is to allow the patient who suffered poor sleep quality self-correct and self-train his/her sleep by staying at home, so as to gradually improve the sleep quality.

For dealing with those issues addressed aforementioned, the present invention suggests a dental oral appliance for using inside human's mouth to assist sleep; the mouth having an upper row of teeth , a lower row of teeth , a tongue , and an upper jaw , with the upper row of teeth and the lower row of teeth defining a lingual side LS; the dental oral appliance comprising a separating portion and a tongue supporting portion; the separating portion defining an upper teeth region at upper part and a lower teeth region at lower part, with the upper teeth region surroundingly adjacent to the upper row of teeth and the lower teeth region surroundingly adjacent to the lower row of teeth ; the tongue supporting portion having an outer edge extendingly connected to the separating portion with respect to one side close to the tongue.

According to an embodiment of the aforesaid dental oral appliance, wherein one end of the tongue supporting portion close to the tongue is located at a higher position relative to the upper teeth region or the separating portion.

According to an embodiment of the aforesaid dental oral appliance, wherein the tongue supporting portion has a connecting portion extended therefrom for adjoining the separating portion, and a distance S is maintained between the connecting portion and the upper row of teeth or the upper jaw .

According to an embodiment of the aforesaid dental oral appliance, wherein the tongue supporting portion has a connecting portion extended therefrom for adjoining the separating portion, and the connecting portion abuts the upper jaw or the lingual side LS of the upper row of teeth.

According to an embodiment of the aforesaid dental oral appliance, wherein the tongue supporting portion gradually slopes downward away from the separating portion.

According to an embodiment of the aforesaid dental oral appliance, wherein the tongue supporting portion has a concave-like curved structure.

According to an embodiment of the aforesaid dental oral appliance, wherein a slot is defined on the tongue supporting portion , with the slot formed in the middle of the tongue supporting portion .

According to an embodiment of the aforesaid dental oral appliance, wherein the separating portion further has a pluralities of touching components disposed on the upper teeth region or lower teeth region, and the touching components are concave or convex structures.

In taking advantage of the present invention, the dental oral appliance of the present invention utilizes a non-invasive and non-surgical method through a self-correction approach, to allow patients suffered from bad sleep quality gradually overcome the sleep issue. Besides, poor-sleep quality patients who suffer from serious snoring, lower tongue position and mouth-breathing issues can adopt this self-regulating/correcting approach to address those issues. This attribute is especially helpful in the field of family healthcare.

For further understanding of the present invention, reference is made to the following detailed description illustrating the embodiments and examples of the present invention. The description is for illustrative purpose only and is not intended to limit the scope of the claim.

### BRIEF DESCRIPTION OF THE DRAWINGS

FIG. 1 is a perspective view of a dental oral appliance of the present invention;

FIG. 2 is a top view of the dental oral appliance of the present invention;

FIG. 3 is a front view of the dental oral appliance of the present invention;

FIG. 4 is a back view of the dental oral appliance of the present invention;

FIG. 5 is a sectional view of the dental oral appliance of the present invention;

FIGS. 6A-6C are schematic views of the dental oral appliance of the present invention in use;

FIG. 7 is a schematic view of the dental oral appliance of another embodiment of the present invention in use;

FIG. 8 is a sectional view of the dental oral appliance of further embodiment of the present invention.

FIG. 9A is a perspective view of the dental oral appliance of further embodiment of the present invention.

FIG. 9B is a top view of the dental oral appliance of further embodiment of the present invention.

FIG. 10A-10B is a perspective view and top view of the dental oral appliance of another embodiment.

### DETAILED DESCRIPTION OF THE PREFERRED EMBODIMENTS

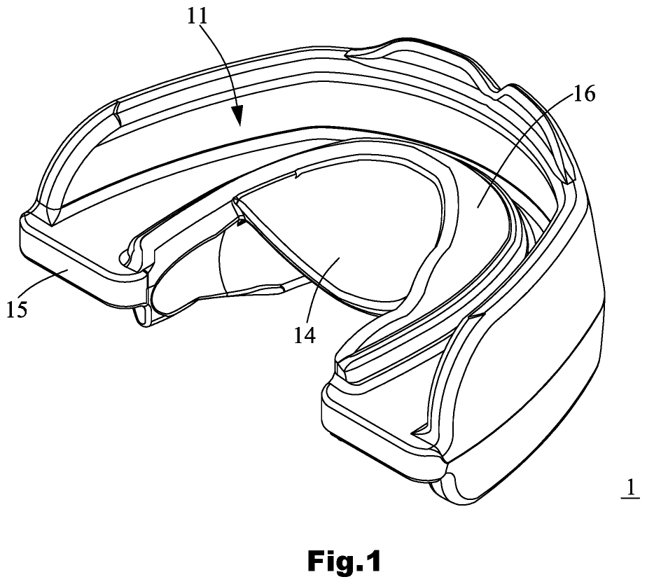

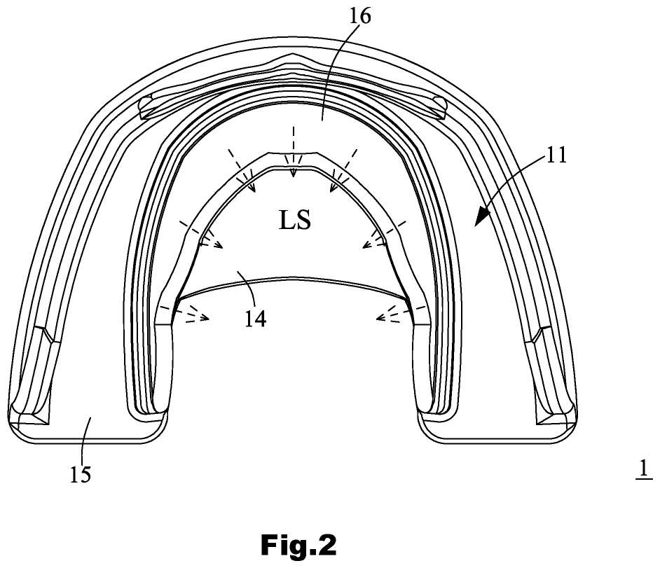

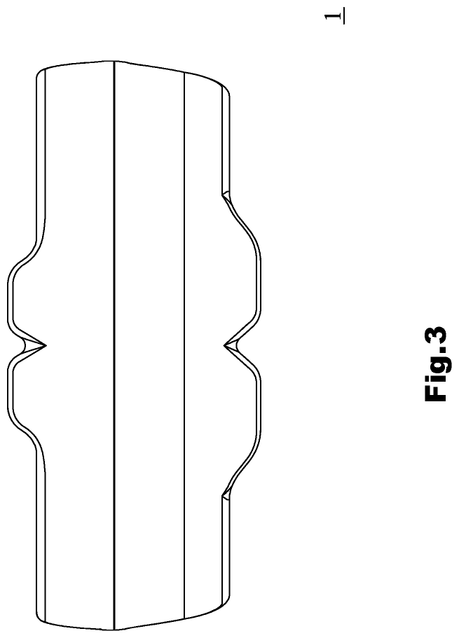

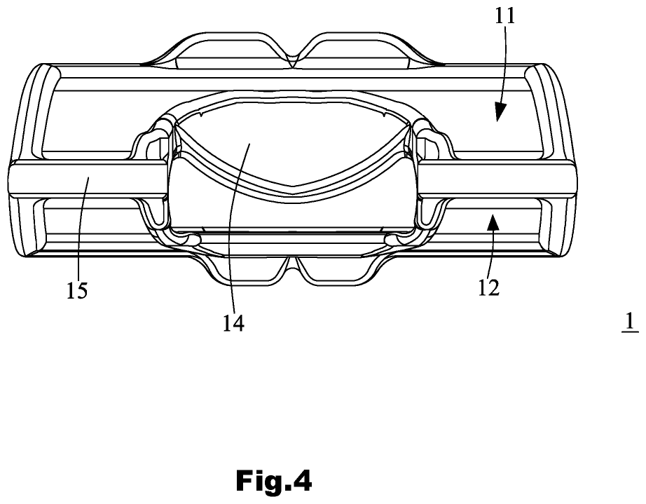

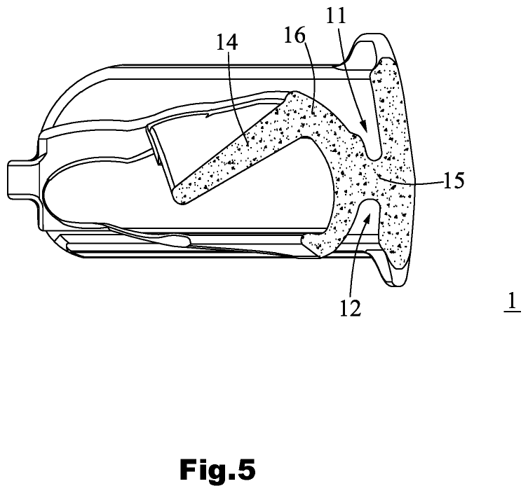

Please refer to FIGS. 1-5, a dental oral appliance 1 of the present invention comprises a separating portion 15, a connecting portion 16, and a tongue supporting portion 14. An upper teeth region 11 is defined above the separating portion 15, and a lower teeth portion 12 is defined thereunder. The outer edge of the tongue supporting portion 14 has the connecting portion 16 extendingly connected to lateral side of the separating portion 15. As shown in FIGS. 1 and 5, the tongue supporting portion 14 extends in a rightward direction to the connecting portion 16. In other words, the tongue supporting portion 14 is abutted to the separating portion 15 through the connecting portion 16. In addition, the tongue supporting portion 14 gradually slopes downward in a direction away from the separating portion 15. That is, the tongue supporting portion 14 dips lower and lower in a leftward direction. Based on the section view of FIG. 5, the tongue supporting portion 14 generally has a slope-like structure. As shown in FIG. 2, when the dental oral appliance 1 is used inside a patient's mouth, a lingual side LS is commonly defined with respect to the tongue of patient. The tongue supporting portion 14 and the connecting portion 16 are both disposed in the lingual side LS of the upper teeth region 11 and lower teeth region 12.

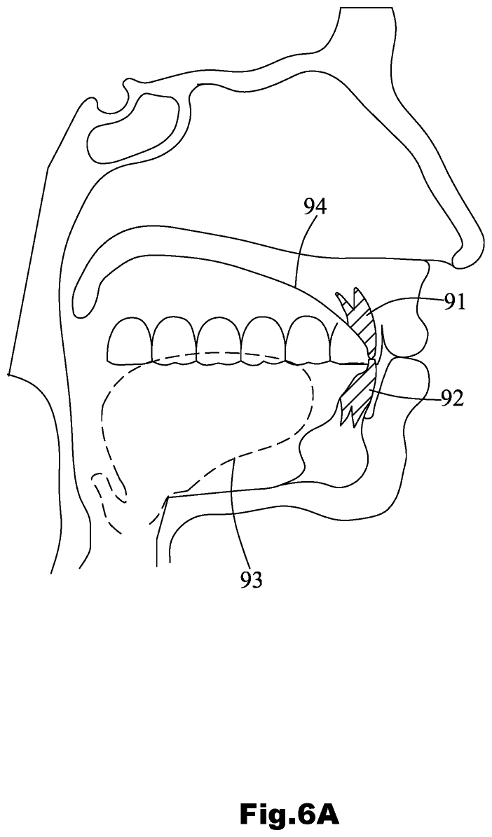

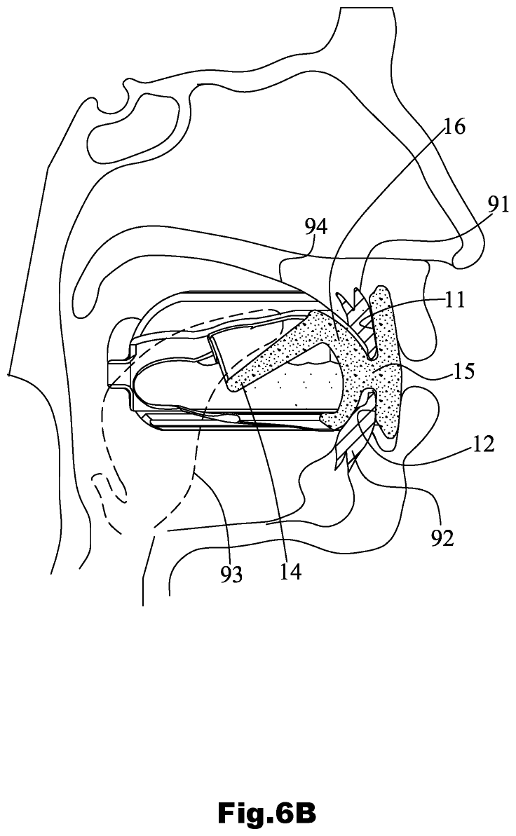

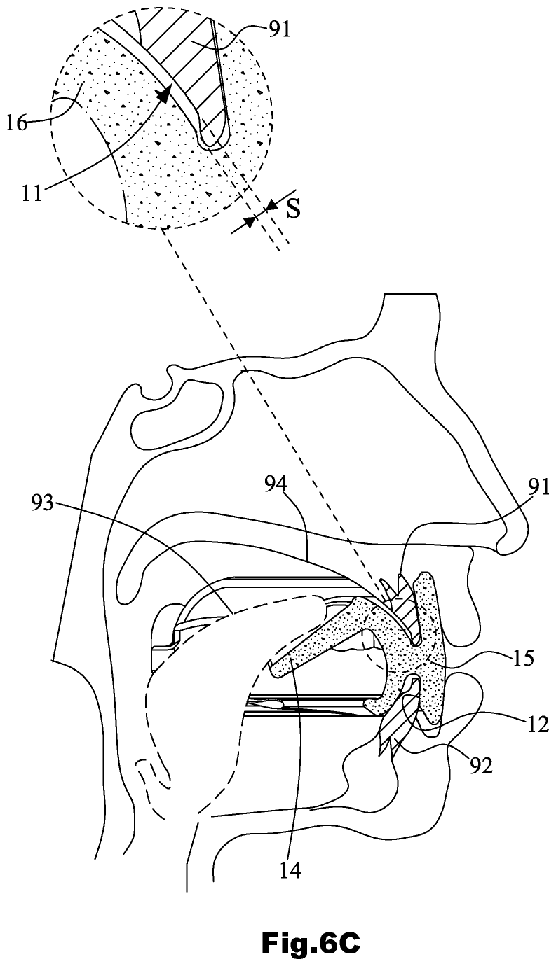

The dental oral appliance 1 of the present invention is designated to assist sleeping and correct snoring. When helping sleep or correcting snore is needed, the dental oral appliance 1 can be disposed in the human's mouth. Please refer to FIGS. 6A-6C, where a patient typically has an upper row of teeth 91, lower row of teeth 92, a tongue 93, and an upper jaw 94. The lingual side LS is defined by the upper row of teeth 91 and lower row of teeth 92 along with the direction towards the tongue 93. When the dental oral appliance 1 is utilized, the upper teeth region 11 is adjacently disposed around the upper row of teeth 92, and the lower teeth region 12 is adjacently disposed around the lower row of teeth 92. The tongue supporting portion 14 is extendingly connected to lateral side of the separating portion 15 and located adjacent to the tongue 93 (i.e., disposed at the lingual side LS direction). As for one of the feature of this embodiment, the free end of the tongue supporting portion 14 next to the tongue 93 (i.e., the left-most end of the tongue supporting portion 14), its position is higher than the separating portion 15.

As shown in the enlarged view of FIG. 6C, a distance S is maintained between the connecting portion 16 and the upper row of teeth 91 or the upper jaw 94. When a patient wears the dental oral appliance 1, the connecting portion 16 does not push against the upper jaw 94. Therefore, the patient does not feel any discomfort and is more likely to wear the dental oral appliance 1 in the long run. However, in other embodiments, the connecting portion 16 is allowed to abut against the upper jaw 94 or the upper row of teeth 91 along the lingual side LS direction. In other works, the distance S is essentially zero, which allows the structure to be worn more secure without loosened, and less likely to be displaced.

Please compare between FIGS. 6A and 6B. When the patient wears the dental oral appliance 1 of the present invention, the upper row of teeth 91 and the lower row of teeth 92 are allowed to bite, so as to grip the position of the dental oral appliance 1, therefore the tongue 93 is able to be placed over the tongue supporting portion 14. By raising the height of the tongue 93, the muscle in the throat can be relaxed and the respiratory tract (i.e. airway) thus can be opened, thereby reducing or eliminating snoring and preventing mouth-breathing caused by lower tongue position. Through gradual adjustment and correction, the dental oral appliance 1 is capable of alleviating snoring symptom and its associated noise. For patients who suffer from bad sleep, the sleep quality of those patients can be gradually improved, only if some trainings and self-adjustment to the dental oral appliance 1 is made; particularly noticed that the training and self-adjustment can be made at home.

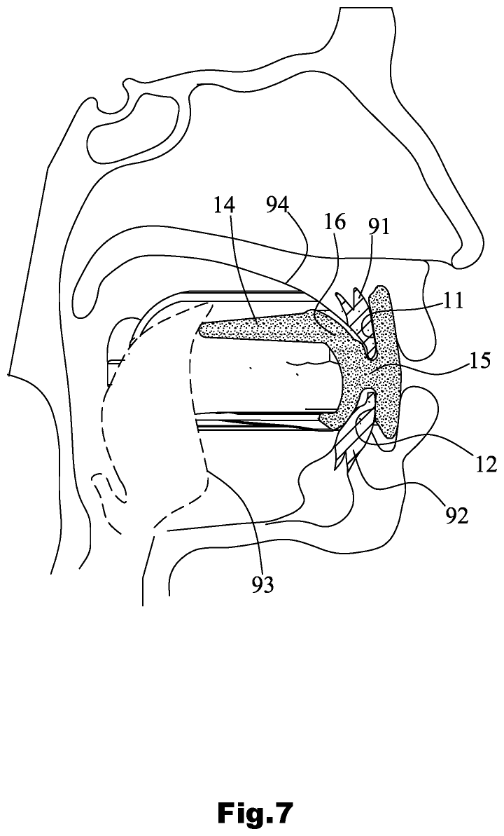

Please refer to FIG. 7, which shows another embodiment of the dental oral appliance 1 of the present invention in use. As shown in this figure, the end of the tongue supporting portion 14 next to the tongue 93 (i.e., the left-most end of the tongue supporting portion 14) is higher than the upper teeth region 11. Thereby, the patient's tongue 93 is pushed to an even higher position, forcing the throat and airway to open up even more.

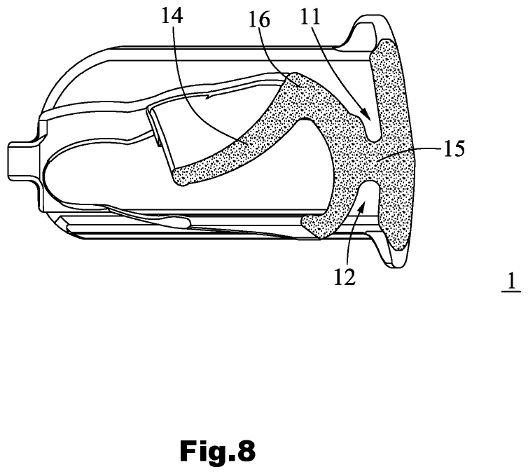

Please refer to FIG. 8, which shows a sectional view of the dental oral appliance of further embodiment of the present invention. As shown in this figure, the tongue supporting portion 14 has a concave-like curved structure. In other words, the curvature along the upper edge of the tongue supporting portion 14 and its curved geometry are capable of mating to the curved geometry along the bottom side of the tongue 93. Therefore, when the tongue 93 is placed over the tongue supporting portion 14, the patient's feeling of discomfort can be essentially eliminated or less severe. Thus, the patient will have an increased desire to use the dental oral appliance 1.

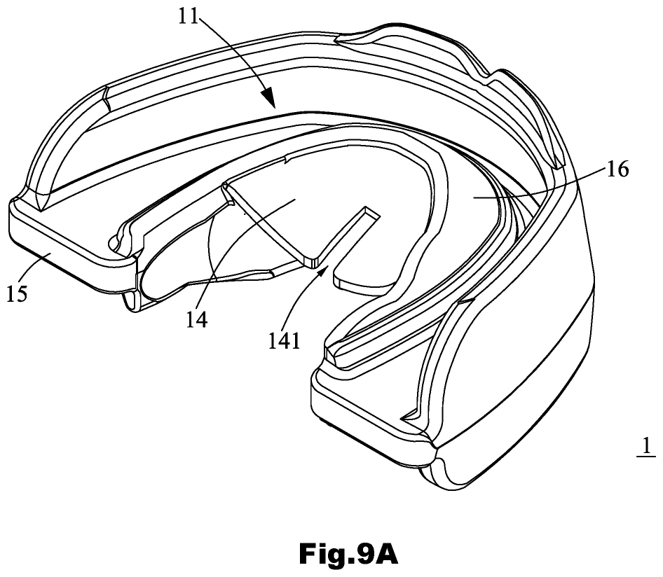

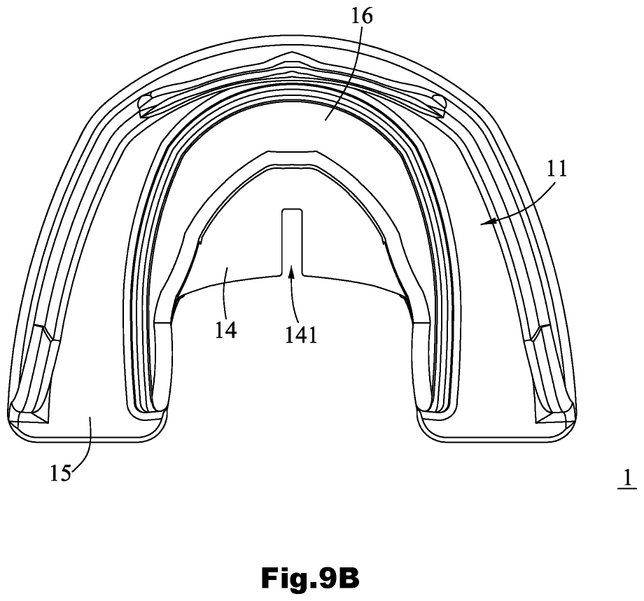

Please refer to FIGS. 9A and 9B, where FIG. 9A is a perspective view of the dental oral appliance of further embodiment of the present invention, and FIG. 9B is a top view thereof. As shown in both figures, a slot 141 is defined in the middle of the tongue supporting portion 14. The slot 141, defined as an opening or a U-shaped structure, is used for receiving the tongue-tie (ankyloglossia) on the bottom of the tongue 93. Thus, when the patient places the tongue 93 over the tongue supporting portion 14, the feeling of discomfort can be reduced while increasing the patient's desire of using the dental oral appliance 1.

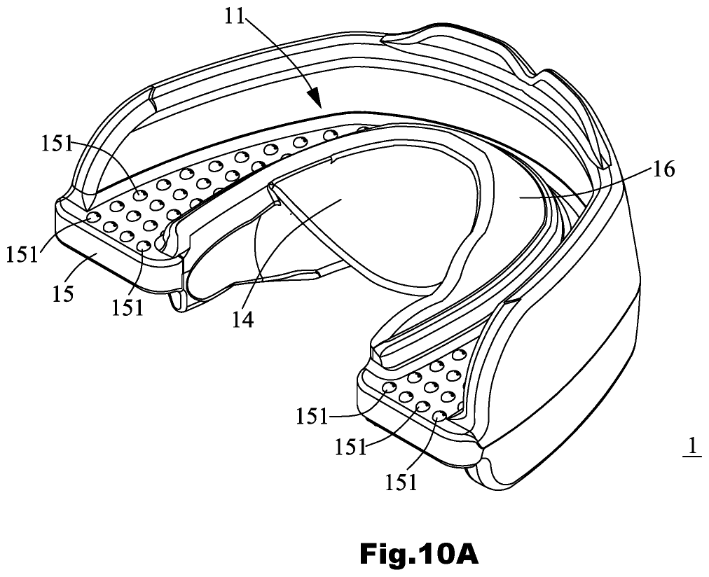

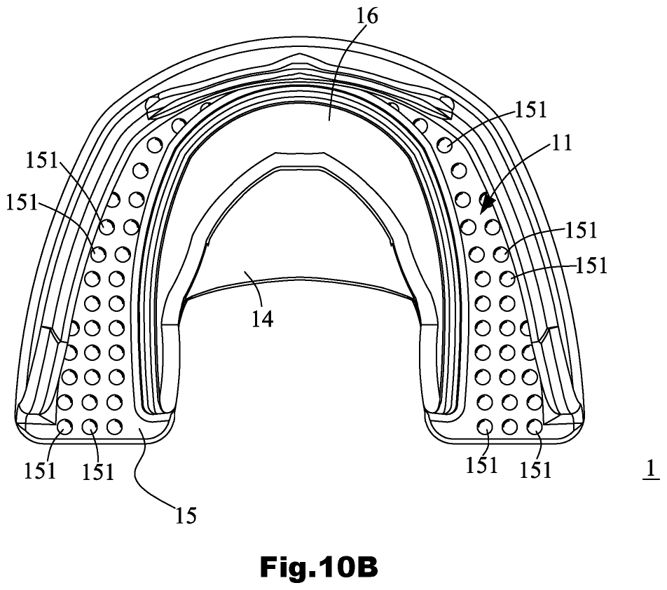

Please refer to FIGS. 10A and 10B, where FIG. 10A-10B is a perspective view and top view of the dental oral appliance of another embodiment. As shown in FIGS. 10A and 10B, the separating portion 15 further has a pluralities of touching components 151 disposed on the upper teeth region 11, and the touching components 151 are convex structures. In this manner, the dental oral appliance 1 can have greater friction to prevent from loosening with respect to the upper row of teeth 91; since the the patient might unconsciously release his/her dental occlusion while falling into sleep, the friction between the upper row of teeth 91 and the upper teeth region 11 will absolutely help the dental oral appliance 1 be kept in right place. Besides, the upper row of teeth 91 may scrape off the upper teeth region 11 in the long run, until the separating portion 15 is broken, therefore the disposal of these convex touching components 151 may retard the rub between the upper row of teeth 91 and the upper teeth region 11.

Similarly, the separating portion 15 can further has a pluralities of touching components 151 disposed at the lower teeth region 12 (not shown), so that the dental oral appliance 1 will have greater friction to prevent from loosening with respect to the lower row of teeth 92, and thus the friction will help the dental oral appliance 1 be kept in right place within the human mouth. Furthermore, the touching components 151 disposed at the lower teeth region 12 may also assist to resist the rub generated from lower row of teeth 92, so that the dental oral appliance 1 can be used for longer time. In the other embodiment, the touching components 151 can be cancave structure, so that the dental oral appliance 1 may also have the same function to be stably held in human mouth without loosening.

Therefore, the dental oral appliance 1 of the present invention utilizes a non-invasive and non-surgical method through a self-correction approach, to allow patients suffering from bad sleep quality to gradually overcome it. In particular, patients who suffer from serious snoring, lower tongue position and mouth-breathing issues can adopt this self-regulating/correcting approach to address those issues. This attribute is especially helpful in the field of family healthcare.

The figures and descriptions supra set forth illustrated the preferred embodiments of the instant disclosure; however, the characteristics of the instant disclosure are by no means restricted thereto. All changes, alternations, combinations or modifications conveniently considered by those skilled in the art are deemed to be encompassed within the scope of the instant disclosure delineated by the following claims.
S: distance
LS: lingual side
1: dental oral appliance
11: upper teeth region
12: lower teeth region
14: tongue supporting portion
141: slot
15: separating portion
151: touching components
16: connecting portion
91: upper row of teeth
92: lower row of teeth
93: tongue
94: upper jaw

What is claimed is:

1. A dental oral appliance (1) for using inside human's mouth to assist sleep, the mouth having an upper row of teeth (91), a lower row of teeth (92), a tongue (93), and an upper jaw (94), with the upper row of teeth (91) and the lower row of teeth (92) defining a lingual side (LS), the dental oral appliance (1) comprising:
a separating portion (15) defining an upper teeth region (11) at upper part and a lower teeth region (12) at lower part, with the upper teeth region (11) surroundingly adjacent to the upper row of teeth (91) and the lower teeth region (12) surroundingly adjacent to the lower row of teeth (92); and
a tongue supporting portion (14) having an outer edge extendingly connected to the separating portion (15) with respect to one side close to the tongue (93).

2. The dental oral appliance (1) as claimed in claim 1, wherein one end of the tongue supporting portion (14) close to the tongue (93) is located at a higher position relative to the upper teeth region (11) or the separating portion (15).

3. The dental oral appliance (1) as claimed in claim 1, wherein the tongue supporting portion (14) has a connecting portion (16) extended therefrom for adjoining the separating portion (15).

4. The dental oral appliance (1) as claimed in claim 3, wherein a distance S is maintained between the connecting portion (16) and the upper row of teeth (91) or the upper jaw (94).

5. The dental oral appliance (1) as claimed in claim 3, wherein the connecting portion (16) abuts the upper jaw (94) or the lingual side (LS) of the upper row of teeth (91).

6. The dental oral appliance (1) as claimed in claim 1, wherein the tongue supporting portion (14) gradually slopes downward away from the separating portion (15).

7. The dental oral appliance (1) as claimed in claim 1, wherein the tongue supporting portion (14) has a concave-like curved structure.

8. The dental oral appliance (1) as claimed in claim 1, wherein a slot (141) is defined on the tongue supporting portion (14), with the slot (141) formed in the middle of the tongue supporting portion (14).

9. The dental oral appliance (1) as claimed in claim 1, wherein the separating portion (15) further has a pluralities of touching components (151) disposed on the upper teeth region (11) or lower teeth region (12).

10. The dental oral appliance (1) as claimed in claim 9, wherein the touching components (151) are concave or convex structures.
